(19) United States
(12) Patent Application Publication
(10) Pub. No.: US 2020/0265945 A1
(43) Pub. Date: Aug. 20, 2020

(54) DEVICE, SYSTEM, AND METHOD FOR OPTIMIZING A HEALTHCARE NETWORK

(71) Applicant: KONINKLIJKE PHILIPS N.V., EINDHOVEN (NL)

(72) Inventors: Reza SHARIFI SEDEH, Malden, MA (US); Yugang JIA, Winchester, MA (US); Daniel Robert ELGORT, New York, NY (US)

(21) Appl. No.: 16/061,401

(22) PCT Filed: Dec. 19, 2016
(86) PCT No.: PCT/EP2016/081676

§ 371 Date: Jun. 12, 2018

Related U.S. Application Data
(60) Provisional application No. 62/270,120, filed on Dec. 21, 2015.

(51) Int. Cl. G16H 40/20; G06Q 10/06; G06Q 10/10; G06Q 50/26

(52) U.S. Cl. CPC G16H 40/20; G06Q 50/26; G06Q 10/10; G06Q 10/06398

(57) ABSTRACT

A device, system, and method optimizes a healthcare network. The method is performed at a device of a healthcare organization, the healthcare organization having a healthcare network including a plurality of healthcare providers. The method includes selecting a healthcare provider to be evaluated. The method includes determining a score for the healthcare provider, the score being based upon at least one of first information relative to the healthcare provider and second information relative to the healthcare network. The method includes generating a recommendation for the healthcare provider based upon the score.

### BACKGROUND INFORMATION

A healthcare organization may be utilized by a patient to receive healthcare from available healthcare providers within a network of the healthcare organization. There are a variety of ways that the healthcare organization may be organized to provide services to the patient. One approach is through an accountable care organization (ACO). The ACO has a network of healthcare providers including primary care physicians (PCP), specialists, etc. The PCP may be in charge of the healthcare plan to be provided to a plurality of patients. Thus, patients may receive treatment from the PCP or from other healthcare providers from within the ACO network.

The ACO may operate as a value-based approach where a bundled-payment or capitation is used in contrast to conventional healthcare organizations that operate in a volume-based approach where a fee for each service is used. Using the value-based approach, the ACO utilizes the healthcare providers who have associated with each other to provide coordinated quality care to the patients. That is, a patient utilizing the ACO may be charged a bundled cost for an overall treatment which may comprise of a plurality of services or treatments. In this manner, the PCP may manage a patient flow and coordinate the patient care within the ACO network.

For an ACO to expand its network for healthcare reasons as well as grow its patient base for financial reasons, the ACO may invite or request PCPs or other healthcare providers who are not currently in the ACO network to join the ACO network. Furthermore, the ACO may already have PCPs or other healthcare providers who are not providing sufficient return for their participation in the ACO network. Because of the value-based approach of the ACO, the ACO may be liable for any and all costs associated with an overall treatment. Accordingly, the ACO must optimize the ACO network of healthcare providers to maximize the patient care while minimizing costs.

### SUMMARY

The present invention is directed to a method comprising: at a device of a healthcare organization, the healthcare organization having a healthcare network including a plurality of healthcare providers: selecting a healthcare provider to be evaluated; determining a score for the healthcare provider, the score being based upon at least one of first information relative to the healthcare provider and second information relative to the healthcare network; and generating a recommendation for the healthcare provider based upon the score.

The present invention is directed to a device of a healthcare organization, the healthcare organization having a healthcare network including a plurality of healthcare providers, comprising: a transceiver communicating via a communications network, the transceiver configured to receive first information relative to a healthcare provider and second information relative to the healthcare network; and a processor selecting the healthcare provider to be evaluated, the processor determining a score for the healthcare provider, the score being based upon at least one of first information relative to the healthcare provider and second information relative to the healthcare network, the processor generating a recommendation for the healthcare provider based upon the score.

The present invention is directed to a non-transitory computer readable storage medium with an executable program stored thereon, wherein the program instructs a microprocessor to perform operations, comprising: selecting a healthcare provider to be evaluated for a healthcare organization, the healthcare organization having a healthcare network including a plurality of healthcare providers; determining a score for the healthcare provider, the score being based upon at least one of first information relative to the healthcare provider and second information relative to the healthcare network; and generating a recommendation for the healthcare provider based upon the score.

### BRIEF DESCRIPTION OF THE DRAWINGS

FIG. 1 shows a system according to the exemplary embodiments.

FIG. 2 shows a server of FIG. 1 according to the exemplary embodiments.

FIG. 3 shows a method for optimizing a healthcare network according to the exemplary embodiments.

### DETAILED DESCRIPTION

The exemplary embodiments may be further understood with reference to the following description and the related appended drawings, wherein like elements are provided with the same reference numerals. The exemplary embodiments are related to a device, a system, and a method for optimizing a healthcare organization. Specifically, the healthcare organization may be an accountable care organization (ACO). In a first aspect, the exemplary embodiments provide a mechanism in which healthcare providers who are not associated with a healthcare network (i.e., out network) of the ACO are evaluated for inclusion in the healthcare network. In a second aspect, the exemplary embodiments provide a mechanism in which healthcare providers who are associated with the healthcare network of the ACO are evaluated to remain in the healthcare network.

The exemplary embodiments relate to providing affordable healthcare to patients via a value-based approach. Specifically, to reduce healthcare costs and improve care, the ACO provides this value-based approach in which a bundled fee is charged to the patient (or a third-party payer) for a given overall treatment to be provided regardless of the steps and treatments that are involved in the course of the overall treatment. The ACO has a healthcare network comprising healthcare providers such as primary care physicians (PCP), specialists, other care providers (e.g., non-doctor provider such as a physical therapist), etc. or healthcare locations such as hospitals, labs, etc. who provide coordinated quality healthcare to the patient (e.g., Medicare patient). Accordingly, in treating a patient, the ACO offers a group of providers that agree on assuming a collective responsibility for delivering and coordinating healthcare for a patient. More generally, the group of providers may agree on the collective responsibility for a designated population (e.g., geographically localized area).

In this process of providing healthcare to a patient of the ACO, the PCP plays an important role in managing a patient flow and coordinating a patient care within the healthcare network of the ACO. Specifically, the PCP may diagnose and/or determine the overall treatment as well as the referrals for treatments involved in the overall treatment to be performed.

Due to the increased effect that the PCP plays in the efficiency of the healthcare network of the ACO, the ACO must intelligently select the PCPs for the healthcare network to improve its financial performance and quality of care. This selection may include adding new PCPs to the healthcare network and eliminating under performing PCPs from the healthcare network. For example, to increase a number of patients who are treated by the ACO (and consequently for financial reasons), the ACO may hire, integrate, request, etc. PCPs who are not currently part of the healthcare network of the ACO (hereinafter referred to as “out network PCPs”) to be included in the healthcare network. Specifically, the ACO may consider including a PCP determined to be competent with many patients into the healthcare network of the ACO (e.g., for healthcare network growth).

Since the ACO is responsible for the overall treatment, associated costs, and outcomes of the patients, the ACO optimizes its financial performance through keeping the patient to only or mostly healthcare providers within the healthcare network of the ACO (without referring the patient to other ACOs, out-network hospitals, out-network specialists, etc.). That is, the Centers for Medicare and Medicaid Services (CMS) hold the ACO responsible for all the associated costs in the overall treatment and outcome of the patient treated by healthcare providers inside and outside the healthcare network of the ACO. Accordingly, the ACO must intelligently organize the ACO and the healthcare network such that an optimal set of PCPs are included/excluded from the ACO to grow the healthcare network of the ACO, minimize patient leakage to out network healthcare providers, and optimize financial performance and clinical outcomes.

The exemplary embodiments are configured to determine the optimal set of PCPs through evaluation of PCPs who are out network PCPs and PCPs who are currently in the healthcare network of the ACO (hereinafter referred to as “in network PCPs”). Therefore, according to a first aspect, the exemplary embodiments may continuously search for the out-network PCPs that bring financial benefits to the ACO and improve the quality of care. Using a variety of sources of information (e.g., claims database of ACO, claims database of CMS, etc.), the exemplary embodiments may evaluate the performance of the out-network CPs. According to a second aspect, the exemplary embodiments may continuously evaluate contributions of the in-network PCPs to the ACO financially and in terms of the quality of healthcare. In this manner, the ACO may identify a least beneficial in-network PCP to subsequently determine an action to be taken for this underperforming in-network PCP (e.g., request improvement or exclusion). Using primarily the claims database of the ACO, the exemplary embodiments may evaluate the performance of the in-network PCPs.

It should be noted that the description herein relates to evaluating PCPs to be included or excluded in the healthcare network of the ACO due to their increased effect on the operation of the ACO network. However, this is only exemplary. The exemplary embodiments may also be utilized for other healthcare providers such as specialists, non-doctor providers, etc. It should also be noted that the description herein relates to optimizing the operation of the ACO. However, this too is only exemplary. The exemplary embodiments may also be utilized in optimizing any healthcare organization, particularly through evaluating the healthcare providers who are included or excluded within the healthcare network thereof.

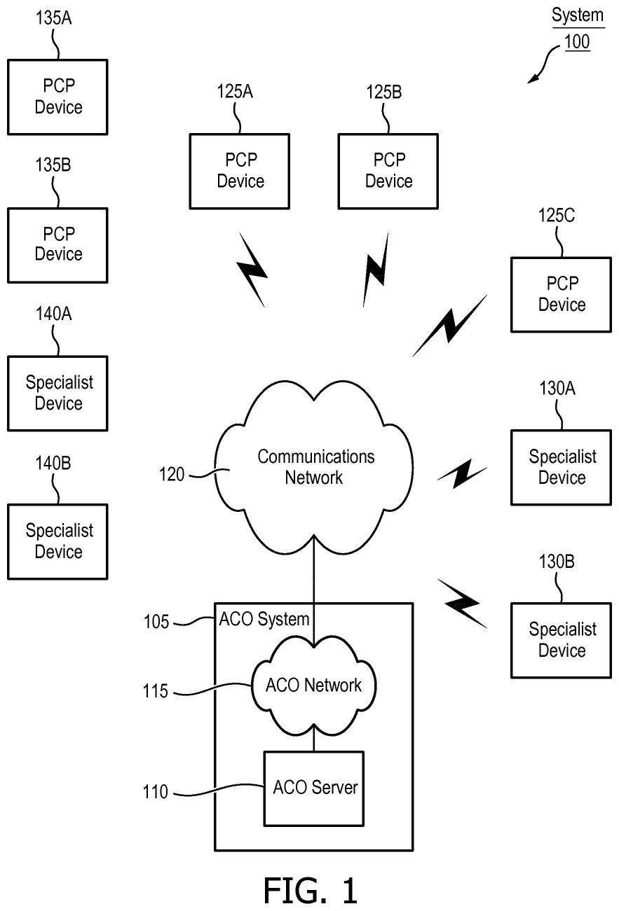

FIG. 1 shows a system 100 according to the exemplary embodiments. The system 100 may relate to a plurality of PCPs who may be associated (e.g., in network PCPs) or unassociated (e.g., out network PCPs) with an ACO healthcare organization. Specifically, the system 100 may include an ACO system 105 in which in network PCPs may utilize the healthcare network associated with the ACO system 105. As will be described in further detail below, the system 100 may include a plurality of in network PCPs using PCP devices 125A-C, a plurality of in network specialists using specialist devices 130A-B, a plurality of out network PCPs using PCP devices 135A-B, and a plurality of out network specialists using specialist devices 140A-B.

The system 100 may include a communications network 120 which is communicatively connected to an ACO network 115 of the ACO system 105. Accordingly, the PCP devices 125A-C and the specialist devices 130A-B utilized by healthcare providers of the healthcare network of the ACO may be authorized to access the ACO system 105 and any data repositories such as a list and description of the healthcare providers included in the healthcare network (e.g., for referral purposes). The communications network 120 may represent any single or plurality of networks used by the PCP devices 125A-C and the specialist devices 130A-B to communicate with the ACO system 105. For example, if the PCP devices 125A-C are computers used at an office, the communications network 120 may include an office network in which the PCP devices 125A-C may initially connect. The office network may connect to a network of an Internet service provider to connect to the Internet. Subsequently, through the Internet, a connection may be established with the ACO network 115. It should be noted that the communications network 120 and all networks that may be included therein may be any type of network. For example, the communications network 120 may be a local area network (LAN), a wide area network (WAN), a virtual LAN (VLAN), a WiFi network, a HotSpot, a cellular network (e.g., 3G, 4G, Long Term Evolution (LTE), etc.), a cloud network, a wired form of these networks, a wireless form of these networks, a combined wired/wireless form of these networks, etc. The communications network 120 may also represent one or more networks that are configured to connect to one another to enable the data to be exchanged among the components of the system 100.

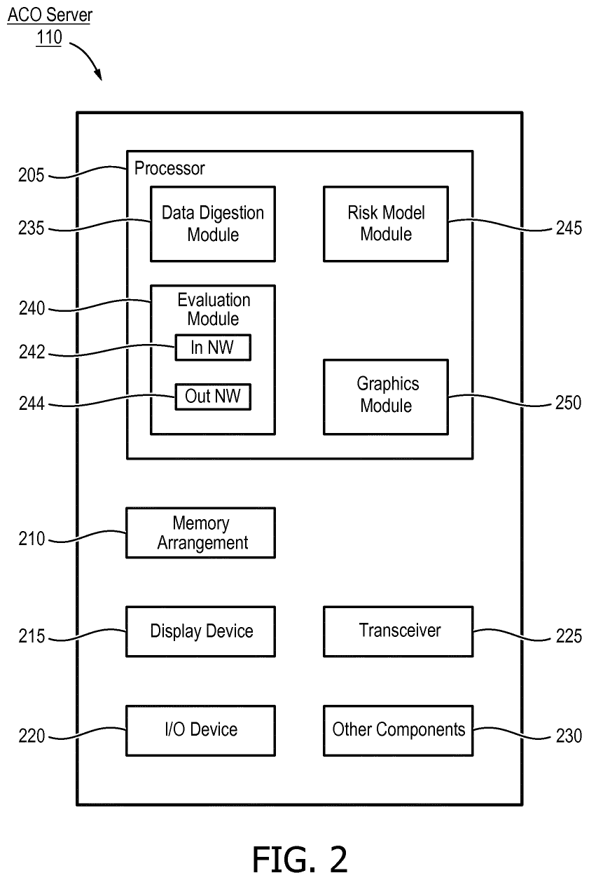

The ACO system 105 may include the ACO network 115 and an ACO server 110. The ACO network 115 of the ACO system 105 may enable the PCP devices 125A-B and the specialist devices 130A-B to access available information provided by the ACO system 105 such as the healthcare network and healthcare providers of the ACO. The ACO network 115 may be configured with an authentication or authorization feature (e.g., an authentication, authorization, and accounting (AAA) procedure (via a AAA server)), that requires identification information to be provided that is used as the basis for granting or denying the access. The ACO network 115 may be a proprietary network using protocols such as the various types described above in a wireless or wired manner. It should be noted that the ACO network 115 may include a variety of components (not shown) to enable these functionalities. For example, the ACO network 115 may include the ACO server 110, data repositories, a router, a switch center, a network management arrangement, etc. The ACO server 110 will be described in further detail below with regard to FIG. 2.

As noted above, the PCP devices 125A-C and the specialist devices 130A-B may be computing devices utilized by healthcare providers who are associated with the healthcare network of the ACO such as in network PCPs. The PCP devices 125A-C and the specialist devices 130A-B may represent any electronic device that is configured to perform the functionalities corresponding to use associated with a healthcare provider. For example, the PCP devices 125A-C and the specialist devices 130A-B may be a portable device such as a tablet, a laptop, etc. or a client stationary device such as a desktop terminal. The PCP devices 125A-C and the specialist devices 130A-B may include the necessary hardware to perform the various procedures and/or treatments as well as the necessary software associated with the procedures/treatments and patient information. The PCP devices 125A-C and the specialist devices 130A-B may also include the required connectivity hardware, software, and firmware (e.g., transceiver) to establish a connection with the communications network 120 to further establish a connection with the ACO network 115.

The system 100 may also represent a localized area. That is, the system 100 may show the PCP devices 125A-C and the specialist devices 130A-B who have agreed upon providing the value-based healthcare treatment to patients of the ACO who are within a defined geographic area. The geographic area may be defined using a variety of factors. For example, the geographic area may be determined for a particular patient and an acceptable distance from the home of the patient. Thus, the PCP devices 125A-C and the specialist devices 130A-B may be determined based upon a specific patient. In another example, the geographic area maybe determined based upon areas designated by an administrator or manager of the ACO. Thus, the PCP devices 125A-C and the specialist devices 130A-B may be selected regardless of the patients. Therefore, the PCP devices 125A-C and the specialist devices 130A-B may be a first group of a plurality of groups of the ACO who have been designated the geographic area. It should be noted that the PCP devices 125A-C and the specialist devices 130A-B may be associated with one or more groups for patients of the ACO for respective geographic areas. It should also be noted that the number of PCP devices 125A-C and the specialist devices 130A-B illustrated in the system 100 of FIG. 1 is only exemplary. Those skilled in the art will understand that there may be any number of PCP devices and specialist devices. In fact, increased PCP devices and specialist devices may ensure that patients of the ACO who are within the defined geographic area may always be treated by healthcare providers who are associated with the healthcare network of the ACO.

The PCP devices 135A-B and the specialist devices 140A-B may be computing devices utilized by healthcare providers who are not associated with the healthcare network of the ACO such as out network PCPs. The PCP devices 135A-B and the specialist devices 140A-B may be substantially similar to the PCP devices 125A-C and the specialist devices 130A-B, respectively. Thus, the PCP devices 135A-B and the specialist devices 140A-B may include the necessary hardware, software, and firmware as described for the in network PCPs and specialists. The PCP devices 135A-B and the specialist devices 140A-B are illustrated in the system 100 of FIG. 1 as not connected to the communications network 120. However, the PCP devices 135A-B and the specialist devices 140A-B may be configured for such a functionality such as connecting to the Internet. As noted above, the PCP devices 135A-B and the specialist devices 140A-B may be utilized by healthcare providers who are not associated with the ACO. Thus, the PCP devices 135A-B and the specialist devices 140A-B may be capable of connecting to the communications network 120 but incapable of connecting to the ACO network 115. However, it should be noted that the ACO network 115 may provide guest access to out network healthcare providers so that the in network healthcare providers may be identified and referred if necessary.

Also substantially similar to the PCP devices 125A- and the specialist devices 130A-B, the PCP devices 135A-B and the specialist devices 140A-B may be healthcare providers who may be included within the defined geographic area. Thus, the PCP devices 135A-B and the specialist devices 140A-B may be considered for inclusion in the healthcare network of the ACO. It should again be noted that the number of PCP devices 135A-C and the specialist devices 140A-B illustrated in the system 100 of FIG. 1 is only exemplary. Those skilled in the art will understand that there may be any number of PCP devices and specialist devices who are out network.

As described above, the ACO server 110 may be a component of the ACO system 105. FIG. 2 shows the ACO server 110 of FIG. 1 according to the exemplary embodiments. The ACO server 110 may provide management and administration functionalities for the ACO system 105. Accordingly, the ACO server 110 may be utilized by a manager or administrator of the ACO. Although the ACO server 110 is described as a network component (specifically a server), the ACO server 110 may be embodied in a variety of ways such as a portable device (e.g., a tablet, a smartphone, a laptop, etc.) or a client stationary device (e.g., a desktop terminal). The ACO server 110 may include a processor 205, a memory arrangement 210, a display device 215, an input and output (I/O) device 220, a transceiver 225, and other components 230 (e.g., an imager, an audio I/O device, a battery, a data acquisition device, ports to electrically connect the ACO server 110 to other electronic devices, etc.).

The processor 205 may be configured to execute a plurality of applications of the ACO server 110. As will be described in further detail below, the processor 205 may utilize a plurality of modules including a data digestion module 235, an evaluation module 240 for healthcare providers who are in network 242 and out network 244, a risk model module 245, and a graphics module 250. The data digestion module 235 may ingest the information used by the other modules. The in network 242 of the evaluation module 240 may determine and evaluate an in network PCP for exclusion from the healthcare network of the ACO while the out network 244 of the evaluation module 240 may determine and evaluate out network PCPs for inclusion in the healthcare network of the ACO. The risk model module 245 may determine whether to adopt the decision of the evaluation module 240 for the ACO. The graphics module 250 may generate a graphical user interface of the evaluation.

It should be noted that the above noted applications and modules each being an application (e.g., a program) executed by the processor 205 is only exemplary. The functionality associated with the applications may also be represented as components of one or more multifunctional programs, a separate incorporated component of the ACO server 110 or may be a modular component coupled to the ACO server 110, e.g., an integrated circuit with or without firmware.

The memory 210 may be a hardware component configured to store data related to operations performed by the ACO server 110. Specifically, the memory 210 may store data related to the ingested information and the healthcare providers who are in network and out network. The display device 215 may be a hardware component configured to show data to a user such as showing the graphical user interface to an administrator or manager while the I/O device 220 may be a hardware component that enables the user to enter inputs. It should be noted that the display device 215 and the I/O device 220 may be separate components or integrated together such as a touchscreen. The transceiver 225 may be a hardware component configured to transmit and/or receive data such as the ingested information. That is, the transceiver 225 may enable the communication with other electronic devices directly or indirectly through the ACO network 115 and/or the communications network 120.

As noted above, the exemplary embodiments may provide a mechanism to determine whether an out network PCP such as those using the PCP devices 135A-B would provide a benefit if included in the healthcare network of the ACO. Specifically, the inclusion of the out network PCP into the healthcare network of the ACO may also invite the patient pool of the out network PCP to increase a financial benefit for the ACO. Additionally, the exemplary embodiments may provide a mechanism to determine whether an in network PCP such as those using the PCP devices 125A-C require a subsequent action to be performed for the improvement of the healthcare network of the ACO. Specifically, the exclusion of the in network PCP from the healthcare network of the ACO may be determined when the in network PCP is determined to have a minimal financial benefit for the healthcare network. Therefore, in performing these evaluations and determinations, the ACO server 110 may utilize the above noted modules.

The data digestion module 235 may ingest information such as the claims information used by the other modules. Specifically, using the transceiver 225, the data digestion module 235 may receive the claims of the ACO as well as the claims of the regional CMS (corresponding to the defined geographic area of the system 100). Accordingly, the ACO claims may be received from data repositories of the ACO system 105 that may be connected to the ACO network 115. The regional CMS claims may be received from data repositories of the CMS (not shown) that may be connected to the communications network 120. The data digestion module 235 may receive the claims information for analysis and formatting to be used by the ACO server 110, particularly the regional CMS claims information which may not be organized in a manner consistent with the ACO server 110. For the other modules such as the evaluation module 240 and the risk model module 245 to perform their respective operations, the data digestion module 235 may receive the regional CMS claims information to understand a performance of out network PCPs and the associated risk of the patient populations and receive the ACO claims information to evaluate the performance of the in network PCPs and understand the capabilities of the healthcare network of the ACO.

It should be noted that the data digestion module 235 may ingest the ACO claims information and the regional CMS claims information at a variety of times. For example, the data digestion module 235 may continuously or at predetermined time intervals update the information such that the information remains up-to-date. In another example, the data digestion module 235 may update the information each time a PCP is to be evaluated (e.g., request the information prior to performing the evaluation). It should also be noted that the ACO claims information may be readily available as this information may be stored in a data repository of the ACO system 105. However, the regional CMS claims information may require a request, an authorization, a fee, etc.

The in network 242 of the evaluation module 240 may determine and evaluate an in network PCP for exclusion from the healthcare network of the ACO. Specifically, using the ACO claims information from the data digestion module 235, the in network 242 of the evaluation module 240 may perform this functionality. The in network 242 of the evaluation module 240 may determine whether the evaluated in network PCP may have a minimal financial benefit or even a detriment to he healthcare network of the ACO (e.g., financially, to a quality of care, a combination thereof, etc.). This determination may warrant the evaluated in network PCP to be excluded from the healthcare network of the ACO or requested to improve performance. For example, the in network 242 of the evaluation module 240 may generate an in network score based upon the ACO claims information. The in network score may be compared to a first in network threshold value that indicates whether the evaluated in network PCP warrants a first action such as requesting an improvement. The in network score may also be compared to a second in network threshold value that indicates whether the evaluated in network PCP warrants a second action such as exclusion from the network.

The out network 244 of the evaluation module 240 may determine and evaluate an out network PCP for inclusion in the healthcare network of the ACO. Specifically, using the ACO claims information as well as the regional CMS claims information from the data digestion module 235, the out network 244 of the evaluation module 240 may perform this functionality. The out network 244 of the evaluation module 240 may determine whether the evaluated out network PCP may off an increased benefit to the network (e.g., financially, to a quality of care, a combination thereof, etc.). In a substantially similar manner as the in network 242 of the evaluation module 240, the out network 244 of the evaluation module 240 may generate an out network score based upon the ACO claims information and the regional CMS claims information. The out network score may be compared to an out network threshold value that indicates whether the evaluated out network PCP should be included in the healthcare network of the ACO. The evaluated out network PCP may be requested to join the healthcare network of the ACO if no request was previously received or accepted if such a request was received.

It is noted that the in network 242 and the out network 244 of the evaluation module 240 may determine the result of the evaluation based upon an exclusive analysis. That is, the result of the evaluation may be formulated on the evaluated PCP alone without consideration of outside factors. Therefore, according to the exemplary embodiments, the ACO server 110 may utilize the results of the evaluation module 240 at face value or upon further analysis.

The risk model module 245 may determine whether to adopt the result of the evaluation module 240 for the ACO. The risk model module 245 may communicate iteratively with the in network 242 and the out network 244 of the evaluation module 240. This communication may be used to evaluate the output or result of the in network 242 and the out network 244 of the evaluation module 240. The risk model module 240 may provide the further analysis to the results. For example, with out network PCPs, the risk model module 240 may consider the patient populations that the evaluated out network PCP work with (using the regional CMS claims database) and the current capabilities of the ACO network (using the ACO claims database). Using this further analysis, the risk model module 245 may accept or reject the result of the evaluation module 240. The risk model module 245 may also request the out network 244 of the evaluation module 240 to provide a further out network PCP to be considered for inclusion if a first result is rejected.

In performing the further analysis, the risk model module 245 may calculate financial risks, clinical risk, benefits of adding or removing an evaluated PCP, etc. For example, the risk model module 245 may utilize data-mining algorithms on the ACO claims information and the regional CMS claims information. Specifically, using the regional CMS claims information, the risk model module 245 may understand the characteristics of the patient populations whereas using the ACO claims information, the risk model module 245 may understand the capabilities of the ACO in dealing with the new patient populations. Thus, the risk model module 245 may accept or reject the result of the evaluation module 240 through these further considerations. For example, the risk model module 245 may reject the result of the out network 244 of the evaluation module 240 when the risk of adding the corresponding patient populations of the evaluated out network PCP to the healthcare network of the ACO is higher than its potential benefits.

In a specific example, the in network or out network score and the further analysis may utilize the claims information. For example, when selecting a PCP (in network or out network), a list of the patients may be retrieved such as through the ACO claims information and/or the regional CMS claims information. The claims associated with each of the patients may be retrieved also from the ACO claims information and/or the regional CMS claims information. The risk model module 245 may determine a risk adjust cost of each claim of a patient and determine a sum of these risks. The risk model module 245 may also perform this operation for the remaining patients of the selected PCP. In this manner, the score (in network or out network) may be a risk adjust cost of all the patients relative to a benchmark cost as defined by, for example, the CMS.

The graphics module 250 may generate a graphical user interface of the evaluation. The graphics module 250 may print out a list of the out network PCPs determined for inclusion in the healthcare network of the ACO as well as a list of the in network PCPs determined for subsequent action.

Using the above modules, the ACO server 110 may be configured to perform the functionalities in determining the healthcare providers of the healthcare network of the ACO. With reference to the system 100 and the PCP devices 125A-C, 135A-B, the ACO server 110 may determine which of the PCP devices 125A-C are to remain in the healthcare network of the ACO and determine which of the PCP devices 135A-B are to be included in the healthcare network of the ACO.

In a first example, the PCP device 135A may be selected for evaluation by the ACO server 110. The selection may have been determined based upon the defined geographic area for the system 100 which may also correspond to, for example, a patient. The data digestion module 235 may have received the ACO claims information and the regional CMS claims information. The regional CMS claims information may indicate the patient pool and risks associated with the selected PCP using the PCP device 135A. The regional CMS claims information may also provide other information regarding the PCP such as quality care related information. The out network 244 of the evaluation module 240 may perform an evaluation analysis for the selected out network PCP utilizing the PCP device 135A. The out network 244 of the evaluation module 240 may determine that the out network PCP utilizing the PCP device 135A may benefit the healthcare network of the ACO (e.g., financially). For example, the out network 244 of the evaluation module 240 may have generated an out network score greater than a predetermined threshold. The risk model module 245 may also perform a further evaluation analysis such as utilizing the ACO claims information in determining how an inclusion of the out network PCP utilizing the PCP device 135A may affect the healthcare network of the ACO (e.g., redundancy in services, satisfy gaps in services, etc.). The risk model module 245 may determine that the out network PCP utilizing the PCP device 135A warrants inclusion to the healthcare network of the ACO and therefore may adopt the result of the out network 244 of the evaluation module 240. Accordingly, the graphics module 250 may generate the list that includes the selected out network PCP.

In a second example, the PCP device 125C may be selected for evaluation by the ACO server 110. The selection may have been determined based upon, for example, an automated alert triggered from a predetermined number of complaints that are lodged, a predetermined time for evaluation, etc. The data digestion module 235 may have received the ACO claims information. The ACO claims information may indicate a satisfaction rating by the patients of the in network PCP utilizing the PCP device 125C as well as other information such as financially related information (e.g., too many referrals to out network specialists). The in network 242 of the evaluation module 240 may perform an evaluation analysis for the selected in network PCP utilizing the PCP device 125C. The in network 242 of the evaluation module 240 may determine that the in network PCP utilizing the PCP device 125C may be detrimental to remain included in the healthcare network of the ACO. For example, the in network 242 of the evaluation module 240 may have generated an in network score lower than a first predetermined threshold. The in network 242 of the evaluation module 240 may also determine whether the in network score is lower than a second, lower predetermined threshold. When the in network score is between the first and second predetermined thresholds, this may indicate that the in network PCP utilizing the PCP device 125C may still improve performance to remain in the healthcare network of the ACO. When the in network score is lower than the second predetermined threshold, this may indicate that the in network PCP utilizing the PCP device 125C is unlikely to be of any more benefit or of significantly high risk to the healthcare network of the ACO. The risk model module 245 may also perform a further evaluation analysis such as utilizing the ACO claims information and/or the regional CMS claims information in determining an overall effect to the healthcare network of the ACO should the in network PCP utilizing the PCP device 125C is to be excluded. The risk model module 245 may determine that the in network PCP utilizing the PCP device 125C warrants exclusion from the healthcare network of the ACO and therefore may adopt the result of the in network 242 of the evaluation module 240. Accordingly, the graphics module 250 may generate the list that includes the selected in network PCP.

It should again be noted that the above description of the mechanism provided by the exemplary embodiments relating to the PCP is only exemplary. Those skilled in the art will understand that the exemplary embodiments may also be utilized with regard to the specialists using the specialist devices 130A-B and 140A-B. For example, the ACO claims information and the regional CMS claims information may indicate that the out network specialist utilizing the specialist device 140B performs a treatment that is not currently covered by any healthcare provider in the healthcare network of the ACO for the defined geographic area. Accordingly, the evaluation analysis may indicate that the inclusion of the specialist utilizing the specialist device 140B may be of particular benefit for inclusion. In this manner, a substantially similar evaluation analysis and recommendation may be generated.

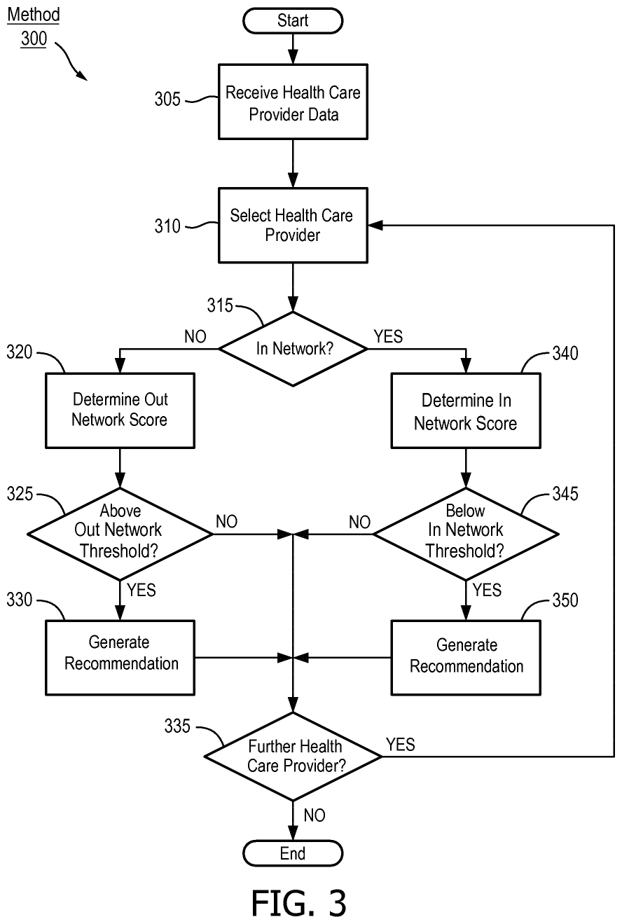

FIG. 3 shows a method 300 for optimizing a healthcare network according to the exemplary embodiments. Specifically, the method 300 may relate to the first and second aspects of managing the healthcare providers in the healthcare network of the ACO via inclusion of out network PCPs and exclusion of in network PCPs. Accordingly, the method 300 may relate to the operations performed by the ACO server 110. The method 300 will be described with regard to the system 100 of FIG. 1 and the ACO server 110 of FIG. 2.

In step 305, the ACO server 110 via the data digestion module 230 receives the healthcare provider data such as the ACO claims information and the regional CMS claims information. As noted above, the healthcare provider data may be received at a variety of different times such as prompted upon a healthcare provider being evaluated. In step 310, the ACO server 110 selects a healthcare provider to be evaluated. As noted above, the PCP may have a significant impact on the healthcare network of the ACO in terms of quality of care and finances.

In step 315, the ACO server 110 determines whether the healthcare provider is in network or out network. With regard to the system 100 of FIG. 1, the PCP devices 125A-C and the specialist devices 130A-B may relate to PCPs and specialists, respectively, who are in network while the PCP devices 135A-B and the specialists devices 140A-B may relate to PCPs and specialists, respectively, who are out network.

If the selected healthcare provider is determined in step 315 to be out network, the ACO server 110 continues the method 300 to step 320. In step 320, the ACO server 110 via the out network 244 of the evaluation module 240 determines an out network score for the selected out network healthcare provider. Specifically, the out network 244 of the evaluation module 240 may utilize the ACO claims information and the regional CMS claims information. The out network score may relate specifically to out network healthcare providers and a potential benefit for the ACO and the healthcare network thereof. In step 320, the ACO server 110 via the risk model module 245 may also perform an evaluation analysis in determining the out network score in relation to the healthcare network of the ACO.

In step 325, the ACO server 110 determines whether the out network score is above an out network threshold. The out network threshold may be indicative of a breakpoint value for the healthcare provider to be a benefit to the ACO and the healthcare network thereof. It is noted that the out network threshold may be fixed, updated, dynamically determined, etc. based upon a variety of factors such as a current standing of the healthcare network and the healthcare providers thereof. If the out network score of the selected out network healthcare provider is below the out network threshold, the ACO server 110 continues the method 300 to step 335. If the out network score of the selected out network healthcare provider is above the out network threshold, the ACO server 110 continues the method 300 to step 330. In step 330, the ACO server 110 generates a recommendation for the inclusion of the selected out network healthcare provider. For example, the graphics module 250 may generate a user interface including the selected out network healthcare provider. Subsequently, the ACO server 110 continues the method 300 to step 335.

In step 335, the ACO server 110 determines whether a further healthcare provider is to be evaluated. For example, each of the out network healthcare providers utilizing the PCP devices 135A-B and specialist devices 140A-B may be evaluated for inclusion in the healthcare network of the ACO. Thus, if a further healthcare provider is to be evaluated, the method 300 returns to step 310.

Returning to step 315, if the selected healthcare provider is determined in step 315 to be in network, the ACO server 110 continues the method 300 to step 340. In step 340, the ACO server 110 via the in network 242 of the evaluation module 240 determines an in network score for the selected in network healthcare provider. Specifically, the in network 242 of the evaluation module 240 may utilize the ACO claims information. The in network score may relate specifically to in network healthcare providers and whether the selected in network healthcare provider remains to be an asset to the ACO and the healthcare network thereof. In step 340, the ACO server 110 via the risk model module 245 may also perform an evaluation analysis in determining the in network score in relation to the healthcare network of the ACO such as using the regional CMS claims information.

In step 345, the ACO server 110 determines whether the out network score is above an in network threshold. The in network threshold may be indicative of a breakpoint value for the healthcare provider to remain a benefit to the ACO and the healthcare network thereof. It is noted that the in network threshold may also be fixed, updated, dynamically determined, etc. based upon a variety of factors such as a current standing of the healthcare network and the healthcare providers thereof. If the in network score of the selected in network healthcare provider is above the out network threshold, the ACO server 110 continues the method 300 to step 335. If the in network score of the selected in network healthcare provider is below the in network threshold, the ACO server 110 continues the method 300 to step 350. In step 350, the ACO server 110 generates a recommendation for a subsequent action to be performed for the selected in network healthcare provider. For example, the subsequent action may be an exclusion of the selected in network healthcare provider from the healthcare network of the ACO. It is noted that the subsequent action may also utilize a plurality of predetermined in network thresholds to also indicate the type of subsequent action to be used (e.g., request an improvement). Subsequently, the ACO server 110 continues the method 300 to step 335.

The exemplary embodiments provide a device, system, and method of evaluating healthcare providers and an association with a healthcare network of a healthcare organization. In evaluating the healthcare providers, according to a first aspect, the exemplary embodiments provide a mechanism to determine whether out network healthcare providers provide a benefit to the healthcare network and the healthcare organization that inclusion of the healthcare provider is recommended. According to a second aspect, the exemplary embodiments provide a mechanism to determine whether in network healthcare providers remain providing a benefit to the healthcare network and the healthcare organization that whether exclusion of the healthcare provider may be determined. In this manner, the exemplary embodiments may optimize the healthcare network of the healthcare organization.

Those skilled in the art will understand that the above-described exemplary embodiments may be implemented in any suitable software or hardware configuration or combination thereof. An exemplary hardware platform for implementing the exemplary embodiments may include, for example, an Intel x86 based platform with compatible operating system, a Windows platform, a Mac platform and MAC OS, a mobile device having an operating system such as iOS, Android, etc. In a further example, the exemplary embodiments of the above described method may be embodied as a computer program product containing lines of code stored on a computer readable storage medium that may be executed on a processor or microprocessor. The storage medium may be, for example, a local or remote data repository compatible or formatted for use with the above noted operating systems using any storage operation.

It will be apparent to those skilled in the art that various modifications may be made in the present disclosure, without departing from the spirit or the scope of the disclosure. Thus, it is intended that the present disclosure cover modifications and variations of this disclosure provided they come within the scope of the appended claims and their equivalent.

1. A method, comprising:
at a device of a healthcare organization, the healthcare organization having a healthcare network including a plurality of healthcare providers:
selecting a healthcare provider to be evaluated;
determining a score for the healthcare provider, the score being based upon at least one of first information relative to the healthcare provider and second information relative to the healthcare network,
wherein the first information is based upon claims information of the healthcare organization and wherein the second information is based upon claims information of a regional governmental health insurance agency; and
generating a recommendation for the healthcare provider based upon the score.

2. The method of claim 1, wherein the healthcare provider is one of an in network healthcare provider and an out network healthcare provider, the in network healthcare provider being associated with the healthcare network, the out network healthcare provider being unassociated with the healthcare network.

3. The method of claim 2, wherein the score is one of an in network score and an out network score, the in network score being used for the in network healthcare provider, the out network score being used for the out network healthcare provider.

4. The method of claim 3, further comprising:
comparing the out network score to an out network threshold value,
wherein the recommendation is to include the out network healthcare provider when the out network score is greater than the out network threshold value.

5. The method of claim 3, further comprising:
comparing the in network score to an in network threshold value,
wherein the recommendation is to exclude the in network healthcare provider when the in network score is lower than the in network threshold value.

6. The method of claim 5, further comprising:
comparing the in network score to a further in network threshold value, the further in network threshold value being greater than the in network threshold value,
wherein the recommendation is to request an improvement in performance by the in network healthcare provider when the in network score is lower than the further in network threshold value and greater than the in network threshold value.

7. The method of claim 1, further comprising:
selecting a further healthcare provider to be evaluated;
determining a further score for the further healthcare provider, the further score being based upon at least one of the first information and the second information; and
generating a further recommendation for the further healthcare provider based upon the further score.

8. The method of claim 7, further comprising:
generating a report including the recommendation for the healthcare provider and the further recommendation for the further healthcare provider.

9. The method of claim 1, wherein the regional governmental health insurance agency is Centers for Medicare and Medicaid Services (CMS).

10. The method of claim 1, wherein the healthcare organization is an accountable care organization (ACO).

11. A device of a healthcare organization, the healthcare organization having a healthcare network including a plurality of healthcare providers, comprising:
a transceiver communicating via a communications network, the transceiver configured to receive first information relative to a healthcare provider and second information relative to the healthcare network; and
a processor selecting the healthcare provider to be evaluated, the processor determining a score for the healthcare provider, the score being based upon at least one of first information relative to the healthcare provider and second information relative to the healthcare network, wherein the first information is based upon claims information of the healthcare organization and wherein the second information is based upon claims information of a regional governmental health insurance agency, the processor generating a recommendation for the healthcare provider based upon the score.

12. The device of claim 11, wherein the healthcare provider is one of an in network healthcare provider and an out network healthcare provider, the in network healthcare provider being associated with the healthcare network, the out network healthcare provider being unassociated with the healthcare network.

13. The device of claim 12, wherein the score is one of an in network score and an out network score, the in network score being used for the in network healthcare provider, the out network score being used for the out network healthcare provider.

14. The device of claim 13, wherein the processor further compares the out network score to an out network threshold value, wherein the recommendation is to include the out network healthcare provider when the out network score is greater than the out network threshold value.

15. The device of claim 13, wherein the processor further compares the in network score to an in network threshold value, wherein the recommendation is to exclude the in network healthcare provider when the in network score is lower than the in network threshold value.

16. The device of claim 15, wherein the processor further compares the in network score to a further in network threshold value, the further in network threshold value being greater than the in network threshold value, wherein the recommendation is to request an improvement in performance by the in network healthcare provider when the in network score is lower than the further in network threshold value and greater than the in network threshold value.

17. The device of claim 11, wherein the processor further selects a further healthcare provider to be evaluated, the processor further determines a further score for the further healthcare provider, the further score being based upon at least one of the first information and the second information, the processor further generates a further recommendation for the further healthcare provider based upon the further score.

18. The device of claim 17, wherein the processor further generates a report including the recommendation for the healthcare provider and the further recommendation for the further healthcare provider.

19. The device of claim 11, wherein the regional governmental health insurance agency is Centers for Medicare and Medicaid Services (CMS).

20. A non-transitory computer readable storage medium with an executable program stored thereon, wherein the program instructs a microprocessor to perform operations, comprising:
selecting a healthcare provider to be evaluated for a healthcare organization, the healthcare organization having a healthcare network including a plurality of healthcare providers;
determining a score for the healthcare provider, the score being based upon at least one of first information relative to the healthcare provider and second information relative to the healthcare network, wherein the first information is based upon claims information of the healthcare organization and wherein the second information is based upon claims information of a regional governmental health insurance agency; and
generating a recommendation for the healthcare provider based upon the score.
